(19) United States
(12) Patent Application Publication
(10) Pub. No.: US 2024/0116991 A1
(43) Pub. Date: Apr. 11, 2024

(54) BILE SALTS BACTOSENSOR AND USE THEREOF FOR DIAGNOSITC AND THERAPEUTIC PURPOSES

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ DE MONTPELLIER, Montpellier (FR)

(72) Inventor: Jérôme BONNET, Montpellier (FR)

(21) Appl. No.: 18/275,331

(22) PCT Filed: Feb. 7, 2022
(86) PCT No.: PCT/EP2022/052877

§ 371 Date: Aug. 1, 2023

(30) Foreign Application Priority Data
Feb. 8, 2021 (EP) 21305165.9

(51) Int. Cl. C07K 14/195; C12N 15/70; G01N 33/68

(52) U.S. Cl. CPC C07K 14/195; C12N 15/70; G01N 33/6893; G01N 2021/6439

(57) ABSTRACT

Bile salts are steroid acids derived from cholesterol in the liver, are released into the gastrointestinal tract to aid in digestion and are thoroughly modified by the resident gut microbiota. Bile acids act as versatile signaling molecules with a variety In of endocrine functions and are linked to several diseases. In particular, serum and urinary bile salts represent biomarkers for early diagnostics of liver dysfunction, yet their current detection methods are impractical and hard to scale. Here the inventors engineered engineered synthetic bile salt receptors using VtrA as sensing domains connected to E. coli CadC system which activates transcription upon dimerization. The performance of the system was assayed for various selection of promoters and they can show that fine tunable response that may be reached by changing expression levels of the bile salt receptor. By performing multiple rounds of directed evolution of the VtrA sensor the inventors obtained a collection of variants with a lower limit of detection and a higher sensitivity. Finally, they show that their bactosensor can detect pathological bile-salt concentrations in samples from patients with liver dysfunction. The present invention thus relates to bile salts bactosensor and use thereof for diagnostic and therapeutic purposes.

### FIELD OF THE INVENTION

The present invention is in the field of medicine, in particular synthetic biology and hepatology.

### BACKGROUND OF THE INVENTION

The liver is a vital organ coordinating metabolic, detoxification, and immunological processes. Liver diseases including hepatitis, cirrhosis, fatty liver disease and cancer are major public health problems and require large-scale screening methods for prevention, diagnosis, and therapeutic monitoring. Liver biopsy and ultrasound-based elastography are the most common methods for the diagnosis and monitoring the progress of liver diseases. However, these technologies are still limited by the requirement of sophisticated infrastructures and well-trained technicians. Liver function can also be monitored by quantifying serum enzymatic activities and bilirubin, but these markers are detectable when damage has already progressed, and are not entirely specific. Liver function is usually monitored by quantifying several enzymatic activities simultaneously due to their lack of specificity. Serum and urinary bile salts are alternative biomarkers for early diagnostics of liver dysfunction, yet their current detection methods are impractical and hard to scale. W02018049362 discloses bile salts sensors, in particular to transcriptional sensor for bile salts in Bacteroides thetaiotaomicron, using bile sensor proteins such as BreR and VFA0359 from Vibrio fischeri.

### SUMMARY OF THE INVENTION

The present invention is defined by the claims. More particularly, the present invention relates to bile salts bactosensor and use thereof for diagnostic and therapeutic purposes.

### DETAILED DESCRIPTION OF THE INVENTION

### Definitions

As used herein, the term “amino acid residue” is intended to include any natural or synthetic amino acid residue, and is primarily intended to indicate an amino acid residue contained in the group consisting of the 20 naturally occurring amino acids, i.e. selected from the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. According to the invention a first amino acid sequence having at least 90% of identity with a second amino acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “fusion protein” refers to a single polypeptide chain having at least two polypeptide domains that are not normally present in a single, natural polypeptide. Thus, naturally occurring proteins are not “fusion proteins”, as used herein. Preferably, a polypeptide of interest (e.g. VtrA) is fused with at least one heterologous polypeptide (e.g. DNA binding domain) via a peptide bond and the fusion protein may also include the linking regions of amino acids between amino acid portions derived from separate proteins.

As used herein, the term “heterologous polypeptide” refers to a polypeptide which does not derive from the same protein to which said heterologous polypeptide is fused.

As used herein, the term “linker” refers to a sequence of at least one amino acid that links the polypeptide of interest to the heterologous polypeptide in the fusion protein. Such a linker may be useful to prevent steric hindrances. Typically a linker comprises 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; or 70 amino acids.

As used herein, the term “VtrA” refers a membrane-bound regulator from Vibrio parahaemolyticus and that is activated via oligomerization. The C-terminal domain of VtrA has been also shown to form a complex with VtrC, which binds to bile salts. Thus, it has been proposed that the VtrA/VtrC complex senses bile salts to activate the DNA-binding domain of VtrA. An exemplary amino acid sequence of VtrA is shown as SEQ ID NO:1 and an exemplary amino acid sequence of VtrC is shown as SEQ ID NO:2.

>tr|Q87GI4|Q87GI4_VIBPA Putative transcriptional
regulator ToxR OS = Vibrio parahaemolyticus
serotype O3:K6 (strain RIMD 2210633) OX = 223926
GN = VPA1332 PE = 1 SV = 1. The transmembrane
domain is indicated in bold, italic and under-
lined. The periplasmic sensing domain is indi-
cated in bold and double underlined.
SEQ ID NO: 1
MTSKKYRIDQKILSSDSPFLISLGSQDRVKLGTHEHLVLLALCEQPGTLL
DKETLIEKGWPGKFVTDSSLTQAIRNIRAHLNDNGKSQKHIKTIAKKGYL
IEKDYVQSLEVIDDKNINETESIRKLVTLTKRNILLISIILQLAFIIYVA
YSYTSIFVSSTAKDDYPSLSFQQDYVYIFSSDFQLSEELGVALINALSAK
EIVPERLYVMLNDKTISFSFISKNKKSKNRVLSTEKKLNYKHISEYIVNE
IEY
> VtrC sequence
SEQ ID NO: 2
MKLNIKRLHLSLTLMSVVMLLVIIYNNFFQPVHFYETSYKYQAADSTYMH
DVAINVSIKGNHFTSDIIIRELVKSENKNYYNVIGHGDIIQKNTHQYYLN
FDNIDVYTGTNKANMKPYKEPTSISSLINKSNNIRVVYLSEEYVVVEFFF
YDGQIITLHRY

As used herein, the term “DNA binding domain” refers to, but is not limited to, a motif that can bind to a specific DNA sequence (e.g., a genomic DNA sequence). DNA binding domains have at least one motif that recognizes and binds to single-stranded or double-stranded DNA. DNA binding domains can interact with DNA in a sequence-specific or a non-sequence-specific manner.

As used herein, the term “CadC transcriptional activator” has its general meaning in the art and refers to the membrane-integrated transcriptional regulator CadC of Escherichia coli. CadC activates expression of the cadBA operon at low external pH with concomitantly available lysine, providing adaptation to mild acidic stress. CadC is a representative of the ToxR-like proteins that combine sensory, signal transduction, and DNA-binding activities within a single polypeptide. Specifically, CadC is composed of a C-terminal periplasmic pH-sensing domain, a single transmembrane helix and an N-terminal cytoplasmic winged helix—turn—helix DNA-binding domain (Buchner S, Schlundt A, Lassak J, Sattler M, Jung K. Structural and Functional Analysis of the Signal-Transducing Linker in the pH-Responsive One-Component System CadC of Escherichia coli. J Mol Biol. 2015 Jul. 31;427(15):2548-61.). CadC dimerizes via its C-terminal periplasmic pH-sensing domain. Thus the expression “E coli CadC transcriptional activator DNA binding domain” refers to the cytoplasmic domain of CadC that is capable of restoring its function via oligomerization of its C-terminal fusion domain.

As used herein, the term “recombinant” refers to an artificial combination of two otherwise separated segments of sequence, e.g., by chemical synthesis or by the manipulation of isolated segments of amino acids or of nucleic acids by genetic engineering techniques.

As used herein, the term “expression cassette” refers to a nucleic acid sequence that is capable in an appropriate setting of driving the expression of a polynucleotide encoding a polypeptide of interest that is incorporated in said expression cassette. When introduced into a host cell, an expression cassette inter alia is capable of directing the cell's machinery to transcribe an incorporated polynucleotide encoding a polypeptide of interest into RNA, which is then usually further processed and finally translated into the polypeptide of interest. The expression cassette can be comprised in an expression vector as will be described in further detail below. The individual elements of the expression cassette according to the present invention are subsequently explained in detail.

As used herein, the term “promoter” refers to a nucleic acid sequence that facilitates the transcription of a polynucleotide of interest. The promoter is operably linked to the polynucleotide of interest. The promoter may also form part of a promoter/enhancer element. Although the physical boundaries between the elements “promoter” and “enhancer” are not always clear, the term “promoter” usually refers to a site on the nucleic acid molecule to which an RNA polymerase and/or any associated factors binds and at which transcription is initiated.

Enhancers potentiate promoter activity, temporally as well as spatially. Many promoters are known in the prior art that are transcriptionally active in a wide range of cell types.

As used herein, the term “operatively linked” refers to a linking between 2 polynucleotides in particular between an expression regulatory sequence (e.g. promoter) and a polynucleotide of interest.

As used herein, the term “vector” refers to an agent that is capable of transferring nucleic acid sequences to target cells (e.g., non-viral vectors, particulate carriers, and liposomes). Typically, “vector construct,” “expression vector, ” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

As used herein, the term “host cell” may be any of a number commonly used cells in the production of exogenous polypeptides or proteins, including prokaryotic host cells.

As used herein the term “probiotic” is meant to designate live microorganisms which, they are integrated in a sufficient amount, exert a positive effect on health, comfort and wellness beyond traditional nutritional effects. Probiotic microorganisms have been defined as “Live microorganisms which when administered in adequate amounts confer a health benefit on the host” (FAO/WHO 2001).

As used herein, the term “transfection” refers to a wide variety of techniques commonly used for the introduction of exogenous DNA into a prokaryotic or eukaryotic host cell, e.g., electroporation, calcium-phosphate precipitation, DEAE-dextran transfection and the like. The host cell may be “transfected” with the vector of the invention by any conventional means known to the skilled artisan. For example transfection may be a transient transfection.

As used herein, the term “bile salt” has its general meaning in the art and are synthesized in the liver from cholesterol, conjugated with glycine or taurine and secreted in bile with cholesterol and lecithin. Exemplary bile salts include the salts of dihydroxy cholic acids, such as deoxycholic acid, glycodeoxycholic acid, taurodeoxycholic acid, chenodeoxycholic acid, glycochenodeoxycholic acid, and taurochenodeoxycholic acid, and trihydroxy cholic acids, such as cholic acid, glycocholic acid, and taurocholic acid. The alkaline salts include sodium, and potassium.

As used herein, the term “output molecule” refers to a polynucleotide or polypeptide that is expressed in response to a particular signal, such as the presence of bile salts.

As used herein, the term “therapeutic polypeptide” refers to any kind of protein or polypeptide exerting a therapeutic action in a subject. The term “therapeutic polynucleotide” refers to any kind of polynucleotide exerting a therapeutic action in a subject.

As used herein, the term “subject” as used herein refers to any mammal organism. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “sample” refers to any volume of a liquid or suspension in which bile salts to be measured can be present in solution.

As used herein, the term “liver dysfunction” “or “hepatic dysfunction” refers to a state in which the liver function is decreased relative to a normal state. Hepatic dysfunction is characteristic of liver diseases.

As used herein, the term “non-alcoholic fatty liver disease” has its general meaning in the art and is intended to refer to the spectrum of disorders resulting from an accumulation of fat in liver cells in individuals with no history of excessive alcohol consumption. In the mildest form, NAFLD refers to hepatic steatosis. The term NAFLD is also intended to encompass the more severe and advanced form non-alcoholic steatohepatitis (NASH), cirrhosis, hepatocellular carcinoma, and virus-induced (e.g., HIV, hepatitis) fatty liver disease.

As used herein, the term “drug-induced liver disease” or “toxic liver injury” is used to describe those instances in which an active agent has caused injury to the liver.

As used herein, the term “alcoholic liver disease” or “alcoholic liver injury” refers to a disease caused by fat accumulation in liver cells caused at least in part by alcohol ingestion. Examples include, but are not limited to, diseases such as alcoholic simple fatty liver, alcoholic steatohepatitis (ASH), alcoholic hepatic fibrosis, alcoholic cirrhosis and the like. It should be noted that alcoholic steatohepatitis is also called alcoholic fatty hepatitis and includes alcoholic hepatic fibrosis.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

As used herein, the term “liver transplantation” has the common meaning in the art and includes partial and whole liver transplantation in which a liver of a donor is partially or wholly resected and partially or wholly transplanted into a recipient. Partial liver transplantation is classified by operation mode into orthotopic partial liver transplantation, heterotopic partial liver transplantation, and the like, and the present invention can be applied to any of them. In partial liver transplantation, a liver transplant or a partial liver transplant from a donor corresponding to about 30-50% of the normal liver volume of a recipient is typically transplanted as a graft into the recipient whose liver has been wholly resected.

As used herein, the term “transplant rejection” as used herein is defined as functional and structural deterioration of the organ due to an active immune response expressed by the recipient, and independent of non-immunologic causes of organ dysfunction. The transplant rejection may be acute or chronic. The term “acute rejection” as used herein refers to a rejection of the transplanted organ developing after the first 5-60 post-transplant days. It is generally a manifestation of cell-mediated immune injury. It is believed that both delayed hypersensitivity and cytotoxicity mechanisms are involved. The immune injury is directed against HLA, and possibly other cell-specific antigens expressed by the tubular epithelium and vascular endothelium. The term “chronic rejection” as used herein refers to a rejection of the transplanted organ developing after the first 30-120 post-transplant days. The term “chronic rejection” also refers to a consequence of combined immunological injury (e.g. chronic rejection) and non-immunological damage (e.g. hypertensive nephrosclerosis, or nephrotoxicity of immunosuppressants like cyclosporine A), taking place month or years after transplantation and ultimately leading to fibrosis and sclerosis of the allograft, associated with progressive loss of kidney function.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “effective amount” refers to a quantity sufficient of the prokaryotic host cell to achieve the beneficial effect.

As used herein, the term “biosensor device” has its general meaning in the art and refers to a device which converts an interaction between a sensor and a recognition molecule into a signal such as an electric signal, so as to measure or detect a target.

As used herein, term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). The DNA targeting endonuclease can be a naturally occurring endonuclease (e.g., a bacterial meganuclease) or it can be artificially generated (e.g., engineered meganucleases, TALENs, or ZFNs, among others). As used herein, the term “TALEN” has its general meaning in the art and refers to a transcription activator-like effector nuclease, an artificial nuclease which can be used to edit a target gene. As used herein, the term “ZFN” or “Zinc Finger Nuclease” has its general meaning in the art and refers to a zinc finger nuclease, an artificial nuclease which can be used to edit a target gene. As used herein, the term “CRISPR-associated endonuclease” has its general meaning in the art and refers to clustered regularly interspaced short palindromic repeats associated which are the segments of prokaryotic DNA containing short repetitions of base sequences.

As used herein, the term “food” refers to liquid (i.e. drink), solid or semi-solid dietetic compositions, especially total food compositions (food-replacement), which do not require additional nutrient intake or food supplement compositions. As used herein the term “food ingredient” or “feed ingredient” includes a formulation which is or can be added to functional foods or foodstuffs as a nutritional supplement. By “nutritional food” or “nutraceutical” or “functional” food, is meant a foodstuff which contains ingredients having beneficial effects for health or capable of improving physiological functions. By “food supplement”, is meant a foodstuff having the purpose of completing normal food diet.

### Polypeptides

The first object of the present invention relates to a fusion protein wherein a “VtrA” polypeptide having an amino acid sequence having at 90% of identity with the amino acid sequence that ranges from the amino acid residue at position 134 to the amino acid residue at position 253 in SEQ ID NO:1 is fused to a DNA binding domain.

In some embodiments, the VtrA polypeptide is fused either directly or via a linker to the DNA binding domain. In some embodiments, the C-terminal end of the DNA binding domain is fused to the N-terminal end of the VtrA polypeptide.

In some embodiments, the DNA binding domain is a E coli CadC transcriptional activator DNA binding domain. In some embodiments, the E coli CadC transcriptional activator DNA binding domain comprises an amino acid sequence having at least 90% of identity with SEQ ID NO:3.

> E. Coli CadC transcription activator DNA binding
domain
SEQ ID NO: 3
MQQPVVRVGEWLVTPSINQISRNGROLTLEPRLIDLLVFFAQHSGEVLSR
DELIDNVWKRSIVTNHVVTQSISELRKSLKDNDEDSPVYIATVPKRGYKL
MVPVIWY

In some embodiments, the VtrA polypeptide is fused to the E coli CadC transcriptional activator DNA binding domain via a linker. In some embodiments, the linker consists of the amino acid sequence as set forth in SEQ ID NO:4.

> Linker
SEQ ID NO: 4
SEEEGEEIMLSSPPPIPEAVPATDSPSHSLNIQNTATPPEQSPVKSKR

In some embodiments, the fusion protein of the present invention consists of an amino acid sequence having at least 90% of identity with the amino acid sequence as set forth in SEQ ID

NO:5.

> CadC DNA binding domain-linker-transmembrane domain-
periplasmic sensing domain
SEQ ID NO: 5

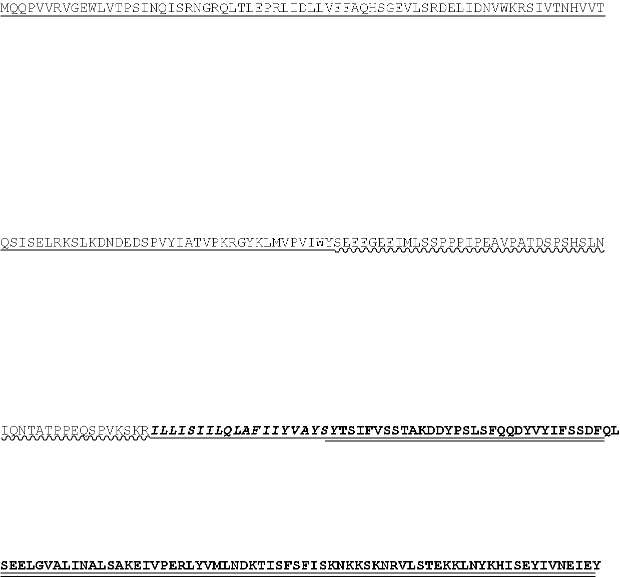

The polypeptides disclosed herein may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, the polypeptides and fusions proteins of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

### Polynucleotides

A further object of the invention relates to a polynucleotide that encodes for a fusion protein of the present invention.

A further object of the present invention relates to an expression cassette comprising the polynucleotide encoding for the fusion protein of the present invention and operably linked thereto control sequences allowing expression in a prokaryotic host cell.

Suitable expression control sequences include promoters that are applicable in the target host organism. Such promoters are well known to the person skilled in the art for diverse hosts from prokaryotic organisms and are described in the literature. For example, such promoters can be isolated from naturally occurring genes or can be synthetic or chimeric promoters. Likewise, the promoter can already be present in the target genome and will be linked to the polynucleotide by a suitable technique known in the art, such as for example homologous recombination.

In some embodiments, the promoter is selected from the group consisting of p14, p10, or p9 promoter having respectively a nucleic acid sequence as set forth in SEQ ID NO:6, SEQ ID NO:7 and SEQ ID NO:8.

>P14
SEQ ID NO: 6
TTGACAATTAATCATCCGGCTCGTATAATGTGTGGA
>P10
SEQ ID NO: 7
TTTCAATTTAATCATCCGGCTCGTATAATGTGTGGA
>p9
SEQ ID NO: 8
TTGCCTCTTAATCATCGGCTCGTATAATGTGTGGA

Expression cassettes according to the invention are particularly meant for an easy to use insertion into target polynucleotides such as vectors or genomic DNA. For this purpose, the expression cassette is preferably provided with nucleotide sequences at its 5′- and 3′-flanks facilitating its removal from and insertion into specific sequence positions like, for instance, restriction enzyme recognition sites or target sequences for homologous recombination as, e.g. catalyzed by recombinases.

### Vectors and Hosts Cells

A further object of the invention relates to vectors, particularly plasmids, cosmids, viruses and bacteriophages used conventionally in genetic engineering, that comprise a polynucleotide or an expression cassette of the present invention.

In some embodiments, the vectors of the present invention are suitable for the transformation of prokaryotic host cells. Methods which are well known to those skilled in the art can be used to construct recombinant vectors. In addition to the polynucleotide or expression cassette of the present invention, the vector may contain further genes such as marker genes which allow for the selection of said vector in a suitable host cell and under suitable conditions. Generally, the vector also contains one or more origins of replication. For genetic engineering, e.g. in prokaryotic host cells, the polynucleotides of the present invention or parts of these molecules can be introduced into plasmids. Expression vectors have been widely described in the literature. As a rule, they contain not only a selection marker gene and a replication origin ensuring replication in the host selected, but also a bacterial promoter and, in most cases, a termination signal for transcription. Between the promoter and the termination signal, there is in general at least one restriction site or a polylinker which enables the insertion of a coding nucleotide sequence. It is possible to use promoters ensuring constitutive expression of the gene and inducible promoters which permit a deliberate control of the expression of the gene. Bacterial promoter sequences possessing these properties are described in detail in the literature. Regulatory sequences for the expression in microorganisms (for instance E. coli) are sufficiently described in the literature. Inducible promoters are also possible. These promoters often lead to higher protein yields than do constitutive promoters.

A further object of the present invention relates to a method for producing a prokaryotic host cell capable of expressing the fusion protein of the invention comprising genetically engineering cells with an above-described polynucleotide, expression cassette or vector of the present invention.

A further object of the present invention relates to a prokaryotic host cell genetically engineered with an above-described polynucleotide, expression cassette or vector of the present invention, and to cells descended from such transformed cells and containing a polynucleotide, expression cassette or vector of the present invention and to cells obtainable by the above-mentioned method for producing the same.

In some embodiments, the prokaryotic host cell is selected among gram-positive or gram-negative bacteria.

In some embodiments, the prokaryotic host cell is selected among non-pathogenic bacteria. In some embodiments, the prokaryotic host cell is selected among bacteria that are derived from a normal internal ecosystem such as bacterial flora. In some embodiments, the prokaryotic host cell is selected among non-pathogenic bacteria that are derived from a normal internal ecosystem of the gastrointestinal tract. Non-limiting examples of nonpathogenic bacteria that are part of the normal flora in the gastrointestinal tract include bacteria from the genera Bacteroides, Clostridium, Fusobacterium, Eubacterium, Ruminococcus, Peptococcus, Peptostreptococcus, Bifidobacterium, Escherichia and Lactobacillus.

In some embodiments, the prokaryotic host cell is selected among anaerobic bacterial cells (e.g., cells that do not require oxygen for growth). Anaerobic bacterial cells include obligate anaerobic cells such as, for example, Bacteroides and Clostridium species. In humans, for example, anaerobic bacterial cells are most commonly found in the gastrointestinal tract.

In some embodiments, the prokaryotic host cell is elected from food grade bacteria. In some embodiments, the prokaryotic host cell is a probiotic.

In some embodiments, the prokaryotic host cell is E. coli.

In some embodiments, the prokaryotic host cell is genetically engineered in such a way that it contains the introduced polynucleotide stably integrated into the genome. The transformation of the prokaryotic host cell with a polynucleotide or vector according to the invention can be carried out by standard methods. For example, calcium chloride transfection is commonly utilized for prokaryotic host cells. The prokaryotic host cell is cultured in nutrient media meeting the requirements of the particular prokaryotic host cell used, in particular in respect of the pH value, temperature, salt concentration, aeration, antibiotics, vitamins, trace elements etc.

In some embodiments, the prokaryotic host cell comprises a polynucleotide that encodes for the VtrC polypeptide having an amino acid sequence as set forth in SEQ ID NO:2. In some embodiments, said polynucleotide is operatively linked to the promoter p5 having the nucleic acid sequence as set forth in SEQ ID NO:9.

> P5 promoter
SEQ ID NO: 9
TTGACAATTAATCATCCGGCTCGTAATTTATGTGGA

In some embodiments, the prokaryotic host cell of the present invention comprises at least one further polynucleotide encoding for an output molecule for which the expression is under the control of the fusion protein of the invention.

In particular, the binding of bile salts to the fusion protein triggers its oligomerization and thus allowing the oligomerization of the CadC transcriptional activator DNA binding domain which can then activate the expression of at least one further polynucleotide encoding for the output molecule that is placed under the control of CadBA promoter.

Accordingly, the prokaryotic host cell of the present invention further comprises a polynucleotide encoding for an output molecule operatively linked to a CadBA promoter. An exemplary nucleic acid for the CadBA promoter is represented by SEQ ID NO:10.

> PCadBA
SEQ ID NO: 10
ATCCATTGTAAACATTAAATGTTTATCTTTTCATGATATCAACTTGCGAT
CCTGATGTGTTAATAAAAAACCTCAAGTTCTCACTTACAGAAACTTTTGT
GTTATTTCACCTAATCTTTAGGATTAATCCTTTTTTCGTGAGTAATCTTA
TCGCCAGTTTGG

In some embodiments, the output molecule is a polypeptide.

In some embodiments, the out molecule is a detection protein that can be detected by biological or physical means.

In some embodiments, the detection protein is a fluorescent protein. The advent of fluorescent proteins has allowed non-invasive intracellular labeling, which are easily detectable by optical means. The green fluorescent protein (GFP) from Aequorea Victoria is now the most widely used reporter gene in many organisms. Multiple variants with different spectral properties have been developed. In some embodiments, the prokaryotic host cell comprises different combinations of fluorescent proteins exhibiting energy transfer provide for differential fluorescence. In some embodiments, the detection protein is selected among luminescent proteins. Certain bacteria (e.g., Vibrio fischeri) have autoinducible luminescent genes that express luciferase, which causes cleaving of luciferin and emission of blue light. Bacteria produce signal molecules, N-acyl homoseine lactones (AELs) that enter bacterial cells and induce transcriptional activation of the genes LuxI, which encodes AHL synthetase, and LuxR, which encodes the AHL-dependent transcriptional activator. A sufficiently high concentration of AHL in the cell causes binding to the LuxR activator and transcription of the luminescence genes.

Alternatively, the detection proteins can be fusion proteins (e.g., green fluorescent protein-Fv) that have a detectable property and that are secreted from the cell. Thus, the secretion can be triggered by bile salts binding to the fusion protein of the present invention. In this case, the detection protein is produced in excess rather than in proportion to the bile salts binding.

In some embodiments, the detection can be performed using RNA aptamers specifically binding a fluorescent probe. Binding of the probe to the aptamer increases its fluorescence and allows detection of gene expression.

In some embodiments, the output molecule is a transcription factor that induces the expression of a detectable molecule or therapeutic molecule. In some embodiments, the output molecule is a repressor factor that represses the expression of a detectable molecule or therapeutic molecule.

In some embodiments, the output molecule is an endonuclease. In some embodiments, the transgene product of interest is an endonuclease that provides for site-specific knock-down of gene function, e.g., where the endonuclease knocks out an allele associated with a genetic disease. For example, where a dominant allele encodes a defective copy of a gene that, when wild-type, is a structural protein and/or provides for normal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the prokaryotic host cell of the present invention can be used to deliver both a site-specific endonuclease that knocks out a defective allele, and can be used to deliver a functional copy of the defective allele, resulting in repair of the defective allele, thereby providing for production of a functional protein. In some embodiments, the DNA targeting endonuclease of the present invention is a TALEN. In some embodiments, the DNA targeting endonuclease of the present invention is a ZFN. In some embodiments, the DNA targeting endonuclease of the present invention is a CRISPR-associated endonuclease. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd or the 4th nucleotide from PAM). The crRNA and tracrRNA can be expressed separately or engineered into an artificial 10 fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector. In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” In some embodiments, the CRISPR-associated endonuclease is a Cpf1 nuclease.

In some embodiments, the output molecule is a therapeutic molecule, in particular a therapeutic polypeptide or a therapeutic polynucleotide.

Therapeutic polypeptides in the sense of the present invention are either proteins, which exist in nature, such as unmodified growth factors, or are designed therapeutic proteins, such as single-chain variable fragments of naturally occurring proteins or variants thereof. Therapeutic polypeptides exert their biological activity via different healing mechanisms. Therapeutic polypeptides are not only growth factors, but also other proteins with biological activity, such as but not limited to protease inhibitors or immune receptor antagonists. Therapeutic polypeptides used in the present innovation can be of form, in amino acid sequence and protein secondary and tertiary structure identical to naturally present, or may be modified or designed for improved action. For example, chimeric proteins can be formed by fusion of different therapeutic polypeptides. Therapeutic polypeptides are also bioactive molecules, not present in nature, such as single chain variable fragments, recombinant antibodies, peptides acting as antagonists, antibodies (e.g. neutralizing antibodies), nanobodies or soluble receptors. In some embodiments, the therapeutic polypeptide is a protein that binds tumor necrosis factor (TNF) or TNF receptors, a protein that binds integrins or integrin receptors, or fibroblast growth factor 19 (FGF19). Examples of proteins that bind TNF or TNF receptors include adalimumumab, certolizumab, golimumab, and infliximab and an anti-TNF Nanobody.

In some embodiments, the output molecule is a polynucleotide, in particular a therapeutic molecule. In some embodiments the output molecule is a ribonucleic acid (RNA). In some embodiments, the output molecule is an interfering RNA (RNAi).

Other kind of output signals include production of pigments via specific operons (like the violacein operon, or the expression of Flavin Mono Oxydase converting tryptophane into indigo), or by the expression of an enzyme which substrate exogenously supplied is transformed in a colorimetric product, like the enzyme Beta-galactosidase and its substrate X-gal for example.

More complex prokaryotic host cells with higher levels of functionality can be created using techniques developed in the field of cellular computation. In these methods, a cell serves as a biochemical computer, processing an input such as bile salts binding using internal logic gates to generate an output. Complex conditional responses to multiple inputs have been engineered for example by implementing AND, NOT, OR, XOR, and IMPLIES logic gates in E. coli cells. For instance, these gates can be implemented using DNA-binding proteins to regulate expression of recombinant vectors. Others systems can be used, such as, but not limited to, recombinase-based logic gates, nucleic acids-based logic gates, or protein-based logic gates. For more information on cellular computing, see R. Weiss, “Cellular Computation and Communications using Engineered Genetic Regulatory Networks,” Ph.D. Thesis, MIT, 2001; M. L. Simpson et al., “Whole-cell biocomputing,” Trends Biotechnol. 19: 317-323 (2001); Yaakov Benenson., «Biomolecular computing systems: principles, progress and potential». «Nature Reviews Genetics, 13(7):455{468, 2012.; Bonnet et al.,“Amplifying genetic logic gates” Science, 340(6132):599{603, 2013. ; Brophy J A N and Voigt C A. «Principles of genetic circuit design ». Nature methods, 11(5):508{520, 2014, all of which are incorporated herein by reference.

### Diagnostic Methods

The prokaryotic host cell of the present invention constitutes whole-cells biosensor (“bactosensor”) that can be suitable for the detection and quantification of bile salts.

Accordingly a further object of the present invention relates to a method for detecting the presence of bile salts in a sample, comprising i) providing at least prokaryotic host cell of the present invention; b) contacting said the prokaryotic host cell with the sample suspected of containing said bile salts for a time sufficient allowing the oligomerization of the fusion proteins binding and then the expression of the detection protein; and c) detecting the expression level of the detection protein wherein the expression level correlated with the amount of the bile salts present in the sample.

In some embodiments, the sample is bodily fluid sample. In some embodiments, the sample is selected from the group consisting of blood samples (including serum or plasma samples), urine samples, cerebrospinal samples, tear samples, saliva samples and synovial samples.

With the method of the present invention, it is possible to measure the concentration of bile salts solved in the sample over a molar range of several orders of magnitude.

The detection protein is assayed for and detected to quantify the bile salts. Typically, when the detection protein is a fluorescent protein, the fluorescence intensity on each cell can be read by methods known in the art such as flow cytometry, laser scanning cytometry, or imaging microscopy. In this way, the fluorescence intensity in all desired wavelength ranges on each individual cell can be detected. The amount or concentration of bile salts in the sample can then be determined using standard methods. In some embodiments, a calibration curve is constructed by measuring the detection protein expression (i.e., its fluorescence) when the cells are combined with samples containing known concentrations of bile salts. As long as a reproducible curve can be constructed, it is not necessary that the response be linear. The measured fluorescence intensity of the detection protein during an assay can then be correlated with the bile salts concentration in the sample using the calibration curve.

It will be appreciated by persons skilled in the art that the method of the present invention may be used in the detection, identification and quantification of bile salts in biological and non-biological samples, such as the diagnosis of disease in medicine or veterinarian science. These applications can be either commercial (in the sense of routine analyses) or serve pure research purposes. Because the method of the present invention may be employed using a virtually limitless variety of modalities, it enables the specific detection of thousands of different bile salts. To the degree that the whole-cell sensors are not destroyed, they may be reusable. In particular, the whole-cell sensors of the present invention are used as a medical diagnostics and disease management in the case of in vitro assays.

In particular, the whole-cell sensor of the present invention is particularly suitable for the diagnosis of a liver dysfunction in a subject.

Accordingly a further object of the present invention relates to a method for determining whether a subject has or is at risk of having a liver dysfunction comprising i) providing at least prokaryotic host cell of the present invention; b) contacting said prokaryotic host cell with a sample obtained from the subject for a time sufficient allowing the oligomerization of the fusion proteins binding and then the expression of the detection protein; and c) detecting the expression level of the detection protein wherein the expression level correlated with the amount of the bile salts present in the sample, and wherein said amount of the bile salts indicates whether the subject has or is at risk of having a liver dysfunction.

A number of acute or chronic pathological conditions lead to liver dysfunction. These include, but are not limited to liver abscess, liver cancer, either primary or metastatic, cirrhosis, such as cirrhosis caused by the alcohol consumption or primary biliary cirrhosis, amebic liver abscess, autoimmune hepatitis, biliary atresia, coccidioidomycosis disseminated, portal hypertension hepatic infections (such as hepatitis A virus, hepatitis B virus, hepatitis C virus, hepatitis D virus, or hepatitis E virus), hemochromatosis, hepatocellular carcinoma, pyogenic liver abscess, Reye's syndrome, sclerosing cholangitis, Wilson's disease, drug induced hepatotoxicity, or fulminant or acute liver failure. In some embodiments, the liver disease is a non-alcoholic fatty liver disease. In some embodiments, the liver disease is a drug-induced liver disease. In some embodiments, the liver disease is an alcoholic liver disease.

In some embodiments, the liver dysfunction may result from a viral infection. The liver is for instance involved in infections by hepatotropic viruses that replicate in the liver and for which the liver is the main target. These include hepatitis A, hepatitis B, hepatitis C, and hepatitis E viruses. In all of these infections, hepatitis and liver dysfunction arise as a consequence of the immune response and reparation mechanisms (e.g. fibrosis) to virus within the liver. In addition, the liver can be affected as part of a generalized host infection with viruses that primarily target other tissues, particularly the upper respiratory tract. Examples of viruses include the herpes viruses (Epstein-Barr virus, cytomegalovirus [CMV], and herpes simplex virus), parvovirus, adenovirus, and severe acute respiratory syndrome (SARS)-associated coronavirus (e.g. SARS-Cov-2).

In some embodiments, the method of diagnosing described herein is applied to a subject who presents symptoms of liver dysfunction without having undergone the routine screening to rule out all possible causes for liver dysfunction. The methods described herein can be part of the routine set of tests performed on a subject who presents symptoms of liver dysfunction such as jaundice, abdominal pain and swelling, swelling in the legs and ankles, itchy skin, dark urine color, pale stool color, bloody color stool, tar-colored stool, chronic fatigue, nausea or vomiting, loss of appetite, tendency to bruise easily. The method of the present invention can be carried out in addition of other diagnostic tools that include ultrasound evaluation (e.g. elastography), biopsy and/or quantification of at least one further biomarkers such as levels of blood AST, ALT, ALP, TTT, ZTT, total bilirubin, total protein, albumin, lactate dehydrogenase, choline esterase and the like.

In some embodiments, the subject underwent a liver transplantation. Accordingly, the present invention is particularly suitable for determining whether a liver transplant subject has or is at risk of having transplant rejection.

In some embodiments, the method of the present invention is particularly suitable for determining whether a subject suffering from a liver disease achieves a response to a therapy.

The method is thus particularly suitable for discriminating responder from non-responder. A responder in the context of the present disclosure refers to a subject that will achieve a response, i.e. a subject who is under remission and more particularly a subject who does not suffer from liver dysfunction. A non-responder subject includes subjects for whom the disease does not show reduction or improvement after the treatment (e.g. the liver dysfunction remains stable or decreases). According to the present invention, the treatment consists in any method or drug that could be suitable for the treatment of liver dysfunction. Some liver problems can be treated with lifestyle modifications, such as stopping alcohol use or losing weight, typically as part of a medical program that includes careful monitoring of liver function. Each liver disease will have its own specific treatment regimen. For example, hepatitis A requires supportive care to maintain hydration while the body's immune system fights and resolves the infection. Patients with gallstones may require surgery to remove the gallbladder. Other diseases may need long-term medical care to control and minimize the consequences of their disease. In patients with cirrhosis and end-stage liver disease, medications may be required to control the amount of protein absorbed in the diet. Other examples include operations required to treat portal hypertension.

The method of the present invention is particularly suitable for monitoring the efficiency of a therapy. Typically a decrease of binding capacity (e.g. between measures performed at different time intervals) indicates that subject does not achieve a response with the therapy. Conversely an increase of binding capacity (e.g. between measures performed at different time intervals) indicates that subject achieves a response with the therapy.

The method of the present is also particularly suitable for evaluating the effects of drugs under development in producing liver injury during a preclinical or clinical studies.

### Biosensors

The whole-cell sensor of the present invention could also be converted into a biosensor device that can be formed using the whole cell sensors of the present invention to be deployed in a microenvironment or microfluidic devices, or a collection of these devices in a multi-chip module or distributed wireless network. The biosensor device can respond to one or more specific chemical and/or physical inputs (e.g. heat or electrical current), generating outputs in the form of detection protein, and communicating with a physical transducer through calorimetric, electrochemical, or preferably fluorescence bioluminescence means. The 1 biosensor may thus comprise a detecting component that comprises the whole-sensor cell and a transducer component for converting a physical change or chemical change generated by the detecting component into an electric signal. According to the mechanism of biomarker detection, there are five types of transducers that may be used in biosensors of the present invention: optical (colorimetric, fluorescent, luminescent, and interferometric) transducers, mass-based (piezoelectric and acoustic wave) transducers, magnetic field based transducers, electrochemical (amperometric, potentiometric and conductometric) transducers, and calorimetric transducers. In some embodiments, the device may comprise additional measuring devices for measuring another parameter of interest. Typically the system comprises additional devices suitable for measuring a physiological phenotype. The physiological phenotype may include physiological parameters such as body temperature, pulse rate, blood pressure, respiratory rate, hydration status and the like. In some embodiments, the system includes an input/output module, an analysis module and a report generation module. The input/output module is configured to receive the amount of bile salts optionally in combinations with additional parameters optionally through the associated communication device. The analysis module is configured to analyze the different parameters that include the amount of bile salts. The report generation module is configured to generate the profile the subject on an analysis of the different parameters. In some embodiments, the system includes a sharing module to share one or more pre-formatted messages with one or more stakeholders based on comparison of the generated profile of the subject. In some embodiments, the system includes providing recommendations to the subject for alerting the subject about the risk of having a liver dysfunction. In some embodiments, the system include enabling transmission of one more messages to an external operator (e.g. a physician) for alerting that the subject has or is at risk of having a liver dysfunction. Thus in some embodiments, the device comprises a communication device. Examples of communication devices include but may not be limited to mobile phones, tablets, desktop computers and the like. Various mediums can be used for connectivity including internet, intranet, Bluetooth, Wi-Fi and the like. In some embodiments the communication device is connected with a server. The measurements of one or more parameters measured by the measuring devices may be indeed transmitted wirelessly to a handheld device comprising a microprocessor. The handheld device may be a smartphone, a tablet device, a cell phone, a mobile internet device, a netbook, a notebook, a personal digital assistant, an internet phone, a holographic device, a holographic phone, a cable internet device, a satellite internet device, an internet television, a DSL internet device and a remote control.

### Kits

A further object of the present invention relates to a kit for performing the methods herein disclosed. The kit comprises one or more plurality of whole-cell sensor as above described and means for determining the expression level of the detection protein. Reagents for particular types of assays can also be provided in kits of the invention. In some embodiments, the kits comprise a device such as a biosensor as described above. In addition, the kits can include various diluents and buffers, labelled conjugates or other agents for the detection of the specifically immunocomplexes. Other components of a kit can easily be determined by one of skill in the art.

### Therapeutic Methods

The prokaryotic host cells are also particularly suitable for therapeutic purposes. In particular, the engineered prokaryotic host cells of the invention are suitable at operating in the gut and would be specifically activated upon arrival in the gut micro-environment in which bile salts are present. Said prokaryotic host cells are particularly suitable for expressing a therapeutic molecule (polynucleotide or polypeptide) in the gut. In presence of bile salts, the expression of the output therapeutic molecule can be triggered.

Thus a further object of the present invention relates to a method of therapy in a subject in need thereof comprising administering to the subject an effective amount of prokaryotic host cell of the invention that comprises a polynucleotide encoding for a therapeutic molecule.

Example of diseases that could be treated by the method of the present invention includes but are not limited to obesity, inflammatory bowel diseases, colorectal cancers, liver diseases and hepatobiliary diseases. In some embodiments, the disease or disorder is peptic ulcer disease, liver cirrhosis, inflammatory bowel disease, an infection, cancer, a vascular disorder, an adverse effect of a medication, or a blood clotting disorder. In some embodiments, the subject has or is at risk of having inflammatory bowel disease. Inflammatory bowel diseases (IBD) refer to a group of inflammatory conditions of the small intestine and colon. In some embodiments, the IBD is Crohn's disease, ulcerative colitis, collagenous colitis, lymphocytic colitis, diversion colitis, Behcet's disease, or indeterminate colitis.

In some embodiments, the prokaryotic host cell is administered into the gut.

In some embodiments, the prokaryotic host cell of the present invention is encapsulated in order to be protected against the stomach. Accordingly, in some embodiments the prokaryotic host cell of the present invention is formulated in compositions in an encapsulated form so as significantly to improve their survival time. In such a case, the presence of a capsule may in particular delay or prevent the degradation of the prokaryotic host cell in the gastrointestinal tract. It will be appreciated that the compositions of the present embodiments can be encapsulated into an enterically-coated, time-released capsule or tablet. The enteric coating allows the capsule/tablet to remain intact (i.e., undissolved) as it passes through the gastrointestinal tract, until such time as it reaches the intestine. Methods of encapsulating live bacterial cells are well known in the art (see, e.g., U.S. patents to General Mills Inc. such as U.S. Pat. No. 6,723,358). For instance, encapsulation can be done with enteric coatings that are preferably methacrylic acid-alkyl acrylate copolymers, such as Eudragit® polymers. Poly(meth)acrylates have proven particularly suitable as coating materials.

In some embodiments, the prokaryotic host cell of the present invention is administered to the subject in the form of a food composition. In some embodiments, the food composition is selected from complete food compositions, food supplements, nutraceutical compositions, and the like. The composition of the present invention may be used as a food ingredient and/or feed ingredient. The food ingredient may be in the form of a solution or as a solid—depending on the use and/or the mode of application and/or the mode of administration. Food and food supplement compositions are for example fermented dairy products or dairy-based products, which are preferably administered or ingested orally one or more times daily. Fermented dairy products can be made directly using the bacteria according to the invention in the production process, e.g. by addition to the food base, using methods known per se. In such methods, the strain(s) of the invention may be used in addition to the micro-organism usually used, and/or may replace one or more or part of the micro-organism usually used. Fermented dairy products include milk-based products, such as (but not limited to) deserts, yoghurt, yoghurt drinks, quark, kefir, fermented milk-based drinks, buttermilk, cheeses, dressings, low fat spreads, fresh cheese, soy-based drinks, ice cream, etc. Alternatively, food and/or food supplement compositions may be non-dairy or dairy non fermented products (e.g. strains or cell-free medium in non-fermented milk or in another food medium). Non-fermented dairy products may include ice cream, nutritional bars and dressings, and the like. Non-dairy products may include powdered beverages and nutritional bars, and the like.

In some embodiments, the food composition that comprises the prokaryotic host cell of the present invention contains at least one prebiotic i.e. a food substance intended to promote the growth of the prokaryotic host cell of the present invention in the intestines. The prebiotic may be selected from the group consisting of oligosaccharides and optionally contains fructose, galactose, mannose, soy and/or inulin; and/or dietary fibers.

In the context of the present invention, the amount of the prokaryotic host cell administered to the subject will depend on the characteristics of the individual, such as general health, age, sex, body weight. The skilled artisan will be able to determine appropriate dosages depending on these and other factors. For example, the prokaryotic host cell shall be able to generate a colony is sufficient to generate a beneficial effect on the subject. If the prokaryotic host cell is administered in the form of a food product, it typically may comprise between 10³ and 10¹² cfu of the prokaryotic host cell of the present invention per g of the dry weight of the food composition.

### Screening Methods

The prokaryotic host cell of the present invention is also particularly suitable for screening purposes. In particular, the prokaryotic host cell of the present invention is particularly suitable for screening of drugs that are suitable e.g. for inhibiting pathogen signalling pathways. In this case, the system is a rewired version of Vibrio parahaemolyticus virulence activation pathway. As it is rebuilt in a non-pathogenic prokaryotic host cell, such as E. coli, and that activation can be monitored easily with the detection of a detectable output molecule, this system could serve as a platform for high-throughput screening of compound libraries to identify new inhibitors (or activators) of Vibrio parahaemolyticus virulence that could be used as therapeutics.

Thus a further object of the present invention relates to a method of screening a plurality of test substances comprising i) contacting a population of prokaryotic host cells of the present invention with said plurality of test substances in presence of an amount of bile salts, and ii) selecting the test substances capable of modulating the expression of the output molecule.

The test substance of the invention may be selected from a library of substances previously synthesised, or a library of substances for which the structure is determined in a database, or from a library of substances that have been synthesised de novo. The test substance may be selected from the group of (a) proteins or peptides, (b) nucleic acids and (c) organic or chemical substances.

In some embodiments, the method comprises the steps consisting of comparing the expression level of the output molecule (e.g. detection protein) with the expression level determined in the absence of the test substance and positively selecting the test substance that provides a decrease or an increase in the expression level of the output molecule.

The invention will be further illustrated by the following figures and examples. However, these examples and figures should not be interpreted in any way as limiting the scope of the present invention.

### FIGURES

FIG. 1. Architecture and mechanism of the VtrAC biosensor. The CadC DBD is fused to the transmembrane and periplasmic domains of VtrA. CadC-VtrA and VtrC are under the control of the P9 and P5 promoters, respectively. Bile salts binding to VtrA/VtrC heterodimeric complexes promote oligomerization of CadC-VtrA and activate downstream expression of the GFP reporter.

FIG. 2. Dose response curve for the VtrAC biosensor. Transfer function of VtrAC-EMeRALD receptor to increasing concentrations of the bile salt taurodeoxycholic acid (TDCA). Experiments are the mean of three experiments performed in triplicates on three different days. Error bars: +/−SD. RPU: reference promoter units.

FIG. 3. Response of the CadC-VtrAC system to different type of bile salts. The activated cells and non-activated cells are further distinguished by gating. The different bile salts used for profiling are labeled in the left-y-axis.

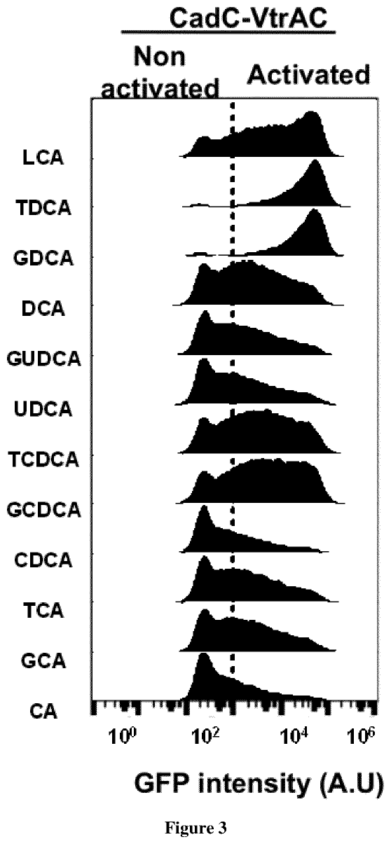

FIG. 4. Bile salt specificity profiles for VtrAC-EMeRALD systems. Cells from an overnight culture were diluted 1:100 in LB containing each bile salt at a 80 uM concentration, and grown for 16 hours at 37° C. before being analyzed by flow cytometry. Values represent the geometric mean of the flow cytometry data. All experiments are the mean of three experiments performed in triplicates on three different days. Error bars: +/−SD. RPU: reference promoter units.

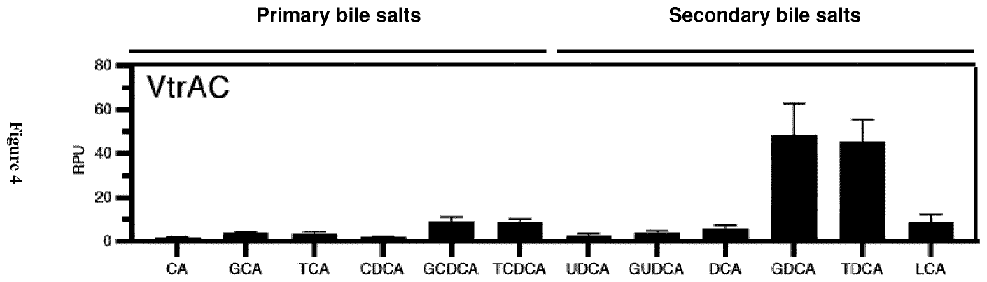

### EXAMPLE

### Material and Methods

### Plasmids and Strains

All constructs were cloned into plasmid J64100_p15A with p15a origin of replication and chloramphenicol resistance gene by isothermal Gibson assembly. All experiments were performed using E. coli strain NEB100μ (New England Biolabs). Plasmids and materials will be made available through Addgene.

Functional Characterization of Synthetic Bile Salt Receptors with SfGFP Fluorescence Outputs

For the experiments of constructs with constitutive promoters, plasmids encoding different constructs were transformed into chemically competent E. coli NEB100μ (New England Biolabs), plated on LB agar plates supplemented with 25 μg/mL chloramphenicol and incubated at 37° C. overnight. For each measurement, three fresh colonies were picked and inoculated into 5 mL of LB/chloramphenicol and grown at 37° C. with vigorous shaking for 16-18 h. In the next day, the cultures were diluted 1:100 into 1 mL of LB/chloramphenicol medium with different concentration of bile salts in 96 deep-well plates (Greiner bio-one), incubated at 37° C. with vigorous shaking for further 4 h and analyzed by flow cytometry. All experiments were performed at least three times in triplicate on three different days. For bile salt specificity profiles, experiments were performed using each bile salt at a 80 μM concentration. For the experiments of constructs with inducible promoters, the overnight cultures were diluted 1:100 into 1 mL of LB/chloramphenicol medium with different concentrations of IPTG, 1.5 mM of benzoic acid, and different concentrations of bile salts in 96 deep-well plates. And then follow the same procedures as constructs with constitutive promoters. All chemicals used in this research were purchased from Sigma-Aldrich.

### Calculation of Relative Promoter Units (RPUs)

Fluorescence intensity measurements among different experiments were converted into RPUs by normalizing them according to the fluorescence intensity of an E. coli strain NEB100μ containing a reference construct and grown in parallel for each experiment. We used the constitutive promoter J23101 and RBS_B0032 as our in vivo reference standard and placed superfolder GFP as a reporter gene in plasmid pSB4K5. We quantified the geometric mean of fluorescence intensity (MFI) of the flow cytometry data and calculated RPUs according to the following equation:

RPU=(MFIₛₐₘₚₗₑ)/(MFI_{referencepromoter})

### Flow Cytometry Analysis

Flow cytometry was performed using an Attune N×T cytometer coupled with high-throughput autosampler (Thermo Fisher Scientific) and Attune N×T™ Version 2.7 Software. In all, 30,000 cells were collected for each data point. Experiment on Attune N×T were performed in 96-well plates with setting; FSC: 200 V, SSC: 380 V, green intensity BL1: 440 V (488 nm laser and a 510/10 nm filter). Flow cytometry data were analyzed using FlowJo 10.0.8r1 (Treestar Inc., Ashland, USA).

### Results

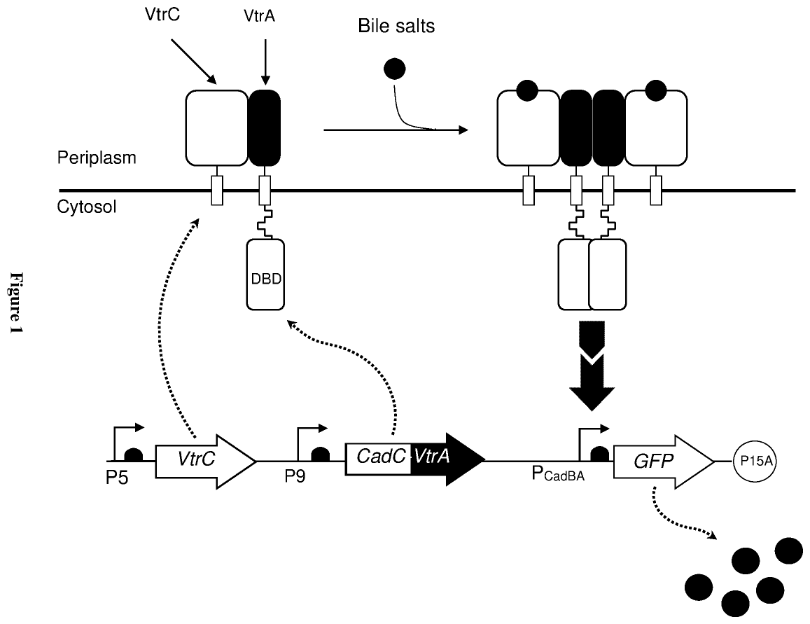

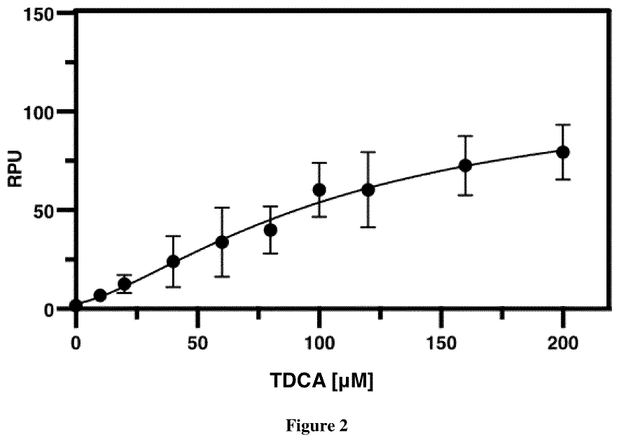

The liver is a vital organ coordinating metabolic, detoxification, and immunological processes. Liver diseases including hepatitis, cirrhosis, fatty liver disease and cancer are major public health problems and require large-scale screening methods for prevention, diagnosis, and therapeutic monitoring. Liver function is usually monitored by quantifying serum enzymatic activities and bilirubin, but these markers are detectable when damage has already progressed, and are not entirely specific. Liver function is usually monitored by quantifying several enzymatic activities simultaneously due to their lack of specificity. Serum and urinary bile salts are alternative biomarkers for early diagnostics of liver dysfunction, yet their current detection methods are impractical and hard to scale. Here we engineered a bacterial biosensor based on non-pathogenic E. coli detecting pathological concentrations of bile salts in clinical samples. We repurposed the bacterial one-component VtrA bile salts sensing domain from Vibrio parahaemolyticus which controls activation of virulence operons when the pathogen enters the gut. We engineered synthetic bile salt receptors using VtrA as sensing domains connected to E. coli CadC system which activates transcription upon dimerization (FIG. 1). We show that our bactosensor can detect bile-salt concentrations in samples (FIGS. 2,3,4). Our work paves the way to a sensitive, scalable, and affordable screening platform for liver dysfunction, that could be deployed in point-of-care or at-home settings and enable large scale monitoring of liver associated diseases. This work also shows how synthetic biology can help address global healthcare challenges while providing tools to decipher and target basic cellular mechanisms, in this case pathogens signaling.

### REFERENCES

Throughout this application, various references describe the state of the art to which this invention pertains. The disclosures of these references are hereby incorporated by reference into the present disclosure.

1. A fusion protein wherein a VtrA polypeptide having an amino acid sequence having at 90% of identity with the amino acid sequence that ranges from the amino acid residue at position 134 to the amino acid residue at position 253 in SEQ ID NO:1 is fused to a DNA binding domain.

2. The fusion protein of claim 1 wherein the DNA binding domain is an E. coli CadC transcriptional activator DNA binding domain that comprises an amino acid sequence having at least 90% identity with SEQ ID NO:3.

3. The fusion protein of claim 1 wherein the VtrA polypeptide is fused either directly or via a linker to the DNA binding domain.

4. The fusion protein of claim 3 wherein the VtrA polypeptide is fused to an Escherichia coli (E. coli) CadC transcriptional activator DNA binding domain via a linker that comprises the amino acid sequence as set forth in SEQ ID NO:4.

5. The fusion protein of claim 1 that comprising an amino acid sequence having at least 90% identity with the amino acid sequence as set forth in SEQ ID NO:5.

6. A polynucleotide that encodes for the fusion protein of claim 1.

7. An expression cassette comprising the polynucleotide of claim 6 and operably linked to a promoter and/or control sequences allowing expression in a prokaryotic host cell.

8. The expression cassette of claim 7 wherein the promoter is selected from the group consisting of p14, p10, or p9 promoter having respectively a nucleic acid sequence as set forth in SEQ ID NO:6, SEQ ID NO:7 and SEQ ID NO:8.

9. A prokaryotic host cell genetically engineered with the polynucleotide of claim 6 or an expression cassette of comprising the polynucleotide.

10. The prokaryotic host cell of claim 9 that is selected from bacteria from the genera Bacteroides, Clostridium, Fusobacterium, Eubacterium, Ruminococcus, Peptococcus, Peptostreptococcus, Bifidobacterium, Escherichia and Lactobacillus.

11. The prokaryotic host cell of claim 9 that is an E. coli host cell.

12. The prokaryotic host cell of claim 9 that comprises a polynucleotide that encodes for the VtrC polypeptide having an amino acid sequence as set forth in SEQ ID NO:2, wherein optionally said polynucleotide is operatively linked to the promoter p5 having the nucleic acid sequence as set forth in SEQ ID NO:9.

13. The prokaryotic host cell of claim 9 that comprises at least one further polynucleotide encoding an output molecule for which expression is under the control of a fusion protein wherein a VtrA polypeptide having an amino acid sequence having at 90% of identity with the amino acid sequence that ranges from the amino acid residue at position 134 to the amino acid residue at position 253 in SEQ ID NO:1 is fused to a DNA binding domain.

14. The prokaryotic host cell of claim 9 that further comprises a polynucleotide encoding an output molecule operatively linked to the CadBA promoter of SEQ ID NO:10.

15. The prokaryotic host cell of claim 14 wherein the output molecule is a detection protein.

16. The prokaryotic host cell of claim 14 wherein the output molecule is a therapeutic polypeptide.

17. A method for detecting the presence of bile salts in a sample, comprising i) providing at least one prokaryotic host cell of claim 15; b) contacting the at least one prokaryotic host cell with the sample suspected of containing said bile salts for a time sufficient to allow expression and oligomerization of fusion proteins encoded by the polynucleotide or the expression cassette and expression of the detection protein; and c) detecting an expression level of the detection protein wherein the expression level correlates with the amount of the bile salts present in the sample.

18. A method for determining whether a subject has or is at risk of having a liver dysfunction comprising i) providing at least one prokaryotic host cell of claim 9; b) contacting said at least one prokaryotic host cell with a sample obtained from the subject for a time sufficient allowing expression and oligomerization of fusion proteins encoded by the polynucleotide or the expression cassette and expression of the detection protein; and c) detecting an expression level of the detection protein wherein the expression level correlates with an amount of bile salts present in the sample, and wherein said amount of bile salts indicates whether the subject has or is at risk of having a liver dysfunction.

19. A method of treating one or more of obesity, an inflammatory bowel disease, a colorectal cancer, a liver disease and a hepatobiliary disease in a subject in need thereof comprising administering to the subject a therapeutically effective amount of the prokaryotic host cell of claim 16.

20. (canceled)

21. A method of screening a plurality of test substances comprising i) contacting a population of prokaryotic host cells of claim 15 with said plurality of test substances in presence of an amount of bile salts, and ii) selecting the test substances capable of modulating the expression of the output molecule.

22. The prokaryotic host cell of claim 15 wherein the detection protein is a fluorescent protein.


SEQUENCE LISTING (10 sequences)

SEQ ID NO: 1 - protein, 253 aa, Vibrio parahaemolyticus
MTSKKYRIDQKILSSDSPFLISLGSQDRVKLGTHEHLVLLALCEQPGTLLDKETLIEKGWPGKFVTDSSLTQAIRNIRAHLNDNGKSQKHIKTIAKKGYLIEKDYVQSLEVIDDKNINETESIRKLVTLTKRNILLISIILQLAFIIYVAYSYTSIFVSSTAKDDYPSLSFQQDYVYIFSSDFQLSEELGVALINALSAKEIVPERLYVMLNDKTISFSFISKNKKSKNRVLSTEKKLNYKHISEYIVNEIEY

SEQ ID NO: 2 - protein, 161 aa, Vibrio parahaemolyticus
MKLNIKRLHLSLTLMSVVMLLVIIYNNFFQPVHFYETSYKYQAADSTYMHDVAINVSIKGNHFTSDIIIRELVKSENKNYYNVIGHGDIIQKNTHQYYLNFDNIDVYTGTNKANMKPYKEPTSISSLINKSNNIRVVYLSEEYVVVEFFFYDGQIITLHRY

SEQ ID NO: 3 - protein, 107 aa, Escherichia coli
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWY

SEQ ID NO: 4 - protein, 48 aa, Escherichia coli
SEEEGEEIMLSSPPPIPEAVPATDSPSHSLNIQNTATPPEQSPVKSKR

SEQ ID NO: 5 - protein, 275 aa, Artificial
  Synthetic CadC DNA binding domain - linker - transmembrane domain - periplasmic sensing domain
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRILLISIILQLAFIIYVAYSYTSIFVSSTAKDDYPSLSFQQDYVYIFSSDFQLSEELGVALINALSAKEIVPERLYVMLNDKTISFSFISKNKKSKNRVLSTEKKLNYKHISEYIVNEIEY

SEQ ID NO: 6 - DNA, 36 bp, Artificial
  Synthetic promoter
ttgacaattaatcatccggctcgtataatgtgtgga

SEQ ID NO: 7 - DNA, 36 bp, Artificial
  Synthetic promoter
tttcaatttaatcatccggctcgtataatgtgtgga

SEQ ID NO: 8 - DNA, 35 bp, Artificial
  Synthetic promoter
ttgcctcttaatcatcggctcgtataatgtgtgga

SEQ ID NO: 9 - DNA, 36 bp, Artificial
  Synthetic promoter
ttgacaattaatcatccggctcgtaatttatgtgga

SEQ ID NO: 10 - DNA, 162 bp, Artificial
  Synthetic promoter
atccattgtaaacattaaatgtttatcttttcatgatatcaacttgcgatcctgatgtgttaataaaaaacctcaagttctcacttacagaaacttttgtgttatttcacctaatctttaggattaatccttttttcgtgagtaatcttatcgccagtttgg